(19) United States
(12) Patent Application Publication
(10) Pub. No.: US 2016/0074575 A1
(43) Pub. Date: Mar. 17, 2016

(54) BOLUS DRUG DELIVERY DEVICE

(71) Applicant: ACACIA, INC., BREA, CA (US)

(72) Inventors: DONGCHUL D. HYUN, BREA, CA (US); ROBERT URENO, BREA, CA (US)

(21) Appl. No.: 14/483,953

(22) Filed: Sep. 11, 2014

(51) Int. Cl. A61M 5/142; A61M 5/14

(52) U.S. Cl. CPC A61M 5/14216; A61M 5/1413; A61M 2005/1405

(57) ABSTRACT

A supplemental pain medication device may be placed in parallel with a basal rate controller for providing a temporary increased level of pain medication to a patient on demand. a trigger of the supplemental pain medication device must be initially depressed in order to allow fluid to flow through the device so that an upper body of the device which is attached to a plunger may slide into a lower body which is attached to a reservoir. When the upper body is slid into the lower body, the plunger also slides into the reservoir to pump fluidic pain medication out of the device.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The various embodiments and aspects disclosed herein relate to a pain medication device.

There are many established benefits that result from delivering pain medication or anesthetic directly to a wound site instead of globally to a patient. Pain medication or anesthetic may be delivered locally by way of a constant slow-release medication mechanism with a catheter embedded in the wound site or the location of the pain. Unfortunately, the level of pain experienced by the patient may increase and decrease over a period of time. When the pain is at its lowest level, the constant slow-release of pain medication may be sufficient for the patient to manage his or her pain. However, when the pain is at its highest level, the constant slow-release of pain medication may be insufficient for the patient to manage his/her pain.

Accordingly, there is a need in the art to provide temporary relief to the patient when he or she is experiencing a high level of pain.

### BRIEF SUMMARY

The various embodiments disclosed herein address the needs discussed above, discussed below and those that are known in the art. In particular, a supplemental pain medication device (e.g., bolus drug delivery device) is shown which may provide fluid flow of pain medication in parallel with a basal rate controller. The basal rate controller is in fluid communication with an elastomeric pump and provides the primary pain medication (e.g., constant flow of pain medication) to a patient. Pain medication may flow through the basal rate controller at a constant rate to the patient. If and when the patient experiences a higher than normal level of pain, the patient and/or medical professional may activate the supplemental pain medication device to provide an increased flow rate of pain medication in addition to the pain medication provided through the basal rate controller in order to relieve the patient of the increased pain being experienced at the time. To activate the supplemental pain medication device, the patient or medical professional depresses a trigger that allows fluidic pain medication to flow through the device. By allowing the pain medication to flow through the device, a plunger is allowed to push fluidic pain medication out of a reservoir and through the device. The trigger is preferably recessed so that the trigger cannot be inadvertently depressed thereby inadvertently enabling the device to provide additional pain medication to the patient. Also, the supplemental pain medication device requires a reset period so that the patient and/or medical professional cannot continuously and indefinitely activate the supplemental pain medication device to provide a sustained increased rate of pain medication to the patient.

More particularly, a device connectable to a pain medication delivery system for providing an additional amount of pain medication fluid to a patient when needed is disclosed. The device may comprise an inlet tube, an outlet tube, a reservoir and a trigger. The inlet tube may be connected to a pressurized supply line for receiving pain medication fluid into the device. The outlet tube may be in fluid communication with the patient for providing pain medication fluid from the device to the patient. The reservoir may be in fluid communication with the inlet tube and the outlet tube. The reservoir may have a container and a plunger. The container may hold a volume of pain medication. The container may be in fluid communication with the inlet tube. The plunger may be traversed between a retracted position and a depressed position. The plunger is operative to flow pain medication fluid out of the outlet tube upon traversal of the plunger to the depressed position from the retracted position. The trigger is positioned between the reservoir and the outlet tube. The trigger may have an actuator traversable between a depressed position and an undepressed position. The trigger may have a flow path aligned to a fluid flow path of the outlet tube to allow the pain medication in the container to flow through the outlet tube when the actuator is in the depressed position. The flow path may be misaligned to the flow path of the outlet tube to stop the pain medication in the container from flowing through the outlet tube when the actuator is in the undepressed position.

The trigger may also further comprise a body having first and second apertures. A connecting tube may be in fluid communication with the container and the first aperture of the body of the trigger. The outlet tube may be in fluid communication with the second aperture of the body of the trigger. The flow path of the trigger may be formed in the actuator. The flow path of the actuator may be aligned to the first and second apertures of the body of the trigger when the actuator is in the undepressed position. The flow path of the actuator may be misaligned to the first and second apertures of the body of the trigger when the actuator is in the depressed position.

The container may be attached to a first body. The plunger may be attached to a second body. The first and second bodies may be capable of being telescoped into each other for sliding the plunger in the container.

The device may further comprise a cap attached to the second body. An exposed surface of the actuator of the trigger may be positioned below an exposed surface of the cap to prevent inadvertent depression of the actuator.

The flow upstream of the reservoir may be more restricted than flow downstream of the reservoir so that upon depression of the plunger into the container, the pain medication fluid in the container flows downstream instead of upstream. By way of example and not limitation, a lumen of the inlet tube may be smaller than a lumen of the connecting tube and the outlet tube for restricting flow upstream of the reservoir more than flow downstream of the reservoir. In another example, a flow restrictor may be mounted upstream of the reservoir to restrict flow upstream of the reservoir more than flow downstream of the reservoir and so that upon depression of the plunger into the container, fluid flows upstream.

In another aspect, a method of operating a secondary device for providing an additional amount of pain medication fluid to a patient when needed is disclosed. The method comprising the steps of connecting the secondary device parallel to a primary pain medication device, the secondary device having zero flow of pain medication fluid until actuated; depressing an actuator of a trigger to establish fluid communication between inlet and outlet tubes to enable flow of additional paid medication fluid out of the secondary device; and depressing first and second bodies with respect to each other to flow pain medication fluid out of the secondary device.

The depressing step may align a flow path of the actuator to a flow of the outlet tube. More generally, the depressing step establishes fluid communication between the inlet and outlet tubes.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of a supplemental pain medication device placed in parallel with an elastomeric pump and a basal rate controller for intermittently providing pain medication to a patient;

FIG. 2 is an enlarged perspective view of the supplemental pain medication device shown in FIG. 1 and a cross-sectional view thereof;

FIG. 3 is a front cross-sectional view of the supplemental pain medication device with a trigger and an upper body in an undepressed position;

FIG. 4 is a front cross-sectional view of the supplemental pain medication device shown in FIG. 4 with the trigger and the upper body in a depressed position;

FIG. 5 is an exploded perspective view of the supplemental pain medication device;

FIG. 6 is a rear view of a half of the upper body of the supplemental pain medication device;

FIG. 6A is a top view of the half of the upper body of the supplemental pain medication device shown in FIG. 6;

FIG. 6B is a cross-sectional view of the half of the upper body of the supplemental pain medication device shown in FIG. 6;

FIG. 6C is a side view of the half of the upper body of the supplemental pain medication device shown in FIG. 6;

FIG. 7 is a front view of a cap disposed on the upper body of the supplemental pain medication device;

FIG. 7A is a cross-sectional view of the cap shown in FIG. 7;

FIG. 7B is a top view of the cap shown in FIG. 7;

FIG. 8 is a rearview of a half of a lower body of the supplemental pain medication device;

FIG. 8A is a top view of the half of the lower body of the supplemental pain medication device shown FIG. 8;

FIG. 8B is a cross-sectional view of the half of the lower body of the supplemental pain medication device shown FIG. 8;

FIG. 8C is a side view of the half of the lower body of the supplemental pain medication device shown in FIG. 8;

FIG. 9 is a front view of a plunger;

FIG. 9A is a top view of the plunger shown in FIG. 9;

FIG. 9B is a cross-sectional view of the plunger shown in FIG. 9;

FIG. 10 is a front view of a reservoir;

FIG. 10A is a top view of the reservoir shown in FIG. 10; and

FIG. 10B is a cross-sectional view of the reservoir shown in FIG. 10.

### DETAILED DESCRIPTION

Referring now to the drawings, a supplemental pain medication device 10 is shown. The device 10 may be connected in parallel with an elastomeric pump 12 and a basal rate controller 14. During use, the basal rate controller 14 provides a constant flow of pain medication to the patient 16. The basal rate controller 14 regulates the flow of pain medication fluid from the elastomeric pump 12 through the basal rate controller 14 and to the patient 16 through tubes 18, 22, 24, 26. The supplemental pain medication device 10 is in fluid communication with tube 18 by way of a bifurcation port 28, tube 30 and bifurcation port 32 and tube 34. The pain experienced by the patient 16 may increase or decrease over a period of time. When the level of pain is low, the pain medication being provided through the basal rate controller 14 may be sufficient to manage the pain experienced by the patient 16. However, when the pain is at its higher range, the pain medication provided through the basal rate controller 14 may be insufficient to manage the pain experienced by the patient 16. The supplemental pain medication device 10 may be manually operated by a person (e.g., patient or medical professional) to inject additional pain medication when the level of pain is high in order to better manage the pain experienced by the patient 16. When the supplemental pain medication device 10 provides that additional amount of pain medication to the patient 16, the flow of pain medication through the basal rate controller 14 preferably is not interrupted (e.g., shut off or decreased) but is maintained at its preset level. Additionally, after providing the additional amount of pain medication to the patient 16, the supplemental pain medication device 10 requires a period of time to reset the device 10 so that the increased level of pain medication cannot be sustained indefinitely. In other words, the patient 16 or medical professional must wait a reset period before providing additional medication to prevent dependency and other medical problems while allowing the patient 16 to manage his or her pain.

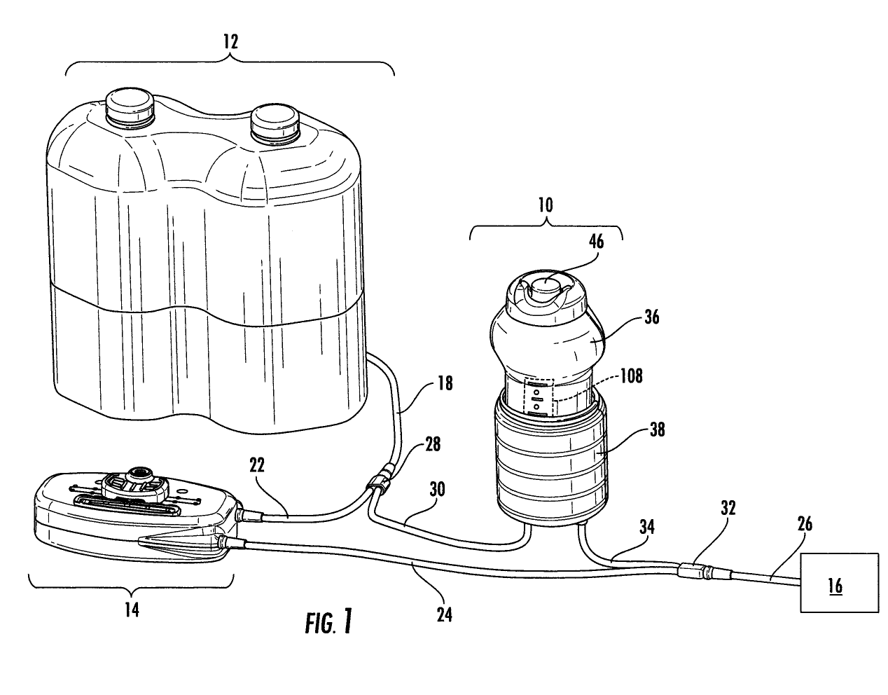

Referring now more particularly to FIGS. 1 and 2, the device 10 may have an upper body 36 and a lower body 38. The upper and lower bodies 36, 38 telescope within one another in order to actuate a plunger 42 into a reservoir 40. When the upper body 36 is telescoped into the lower body 38, the plunger 42 moves within the reservoir 40 to pump the pain medication contained within the inner cavity 44 of the reservoir 40 out of the supplemental pain medication device 10 and through the tube 34, into the tube 26 and into the patient 16. As the plunger 42 is being depressed downward further into the reservoir 40, the flow of pain medication through the basal rate controller 14 preferably remains constant so that the total flow rate of pain medication to the patient 16 is a sum of the flow rate of pain medication through the basal rate controller 14 and out of the supplemental pain medication device 10. In this manner, the patient 16 can actuate the supplemental pain medication device 10 to provide increased levels of pain medication when the pain level is high.

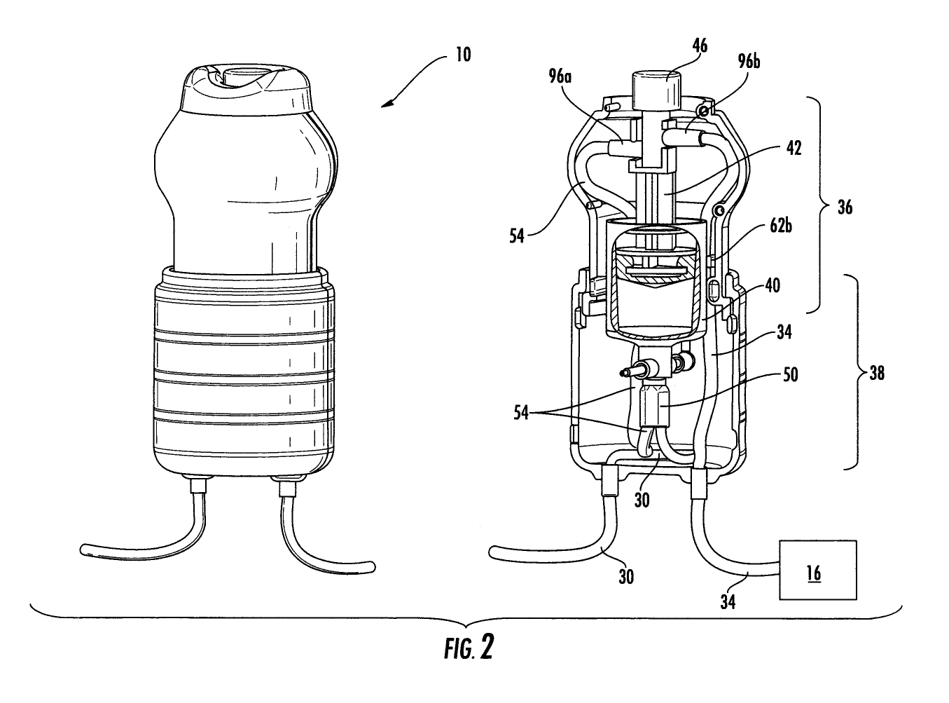

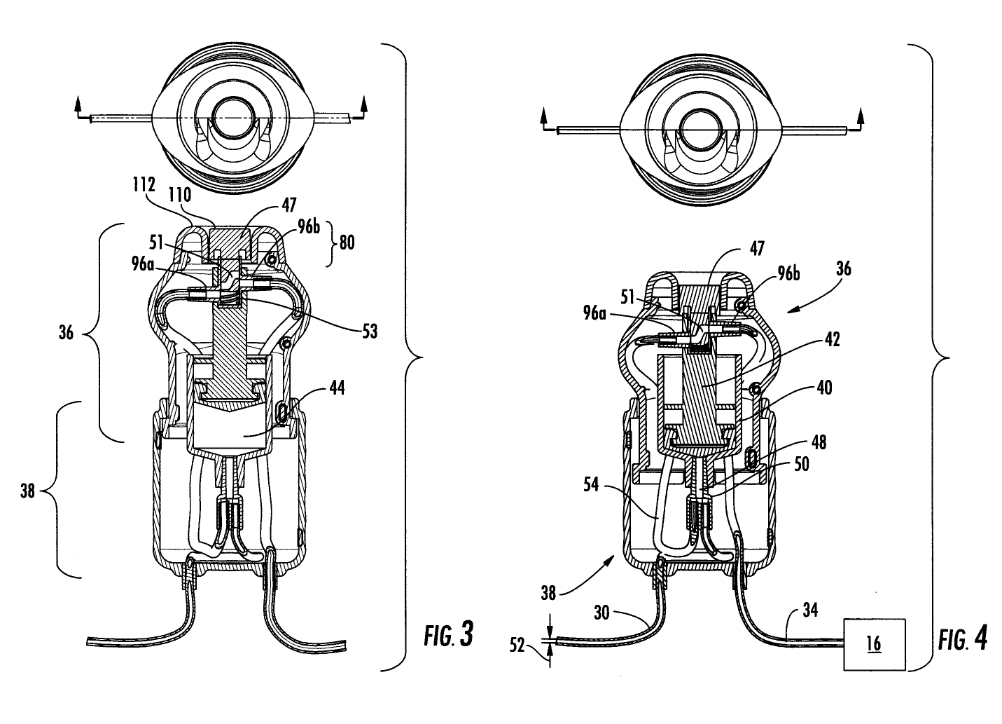

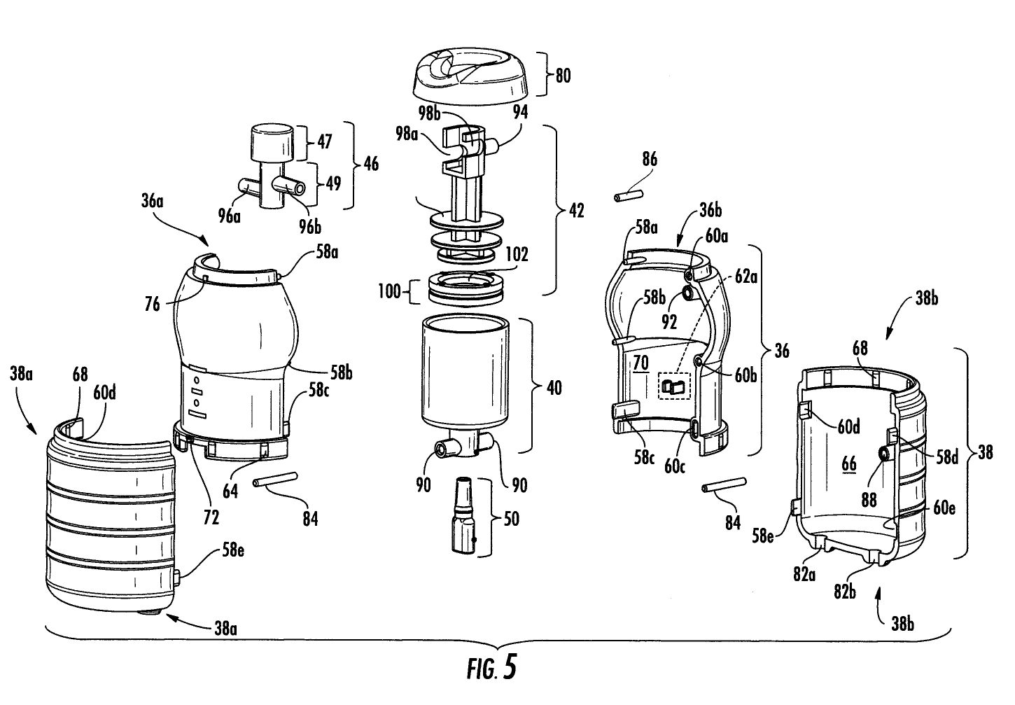

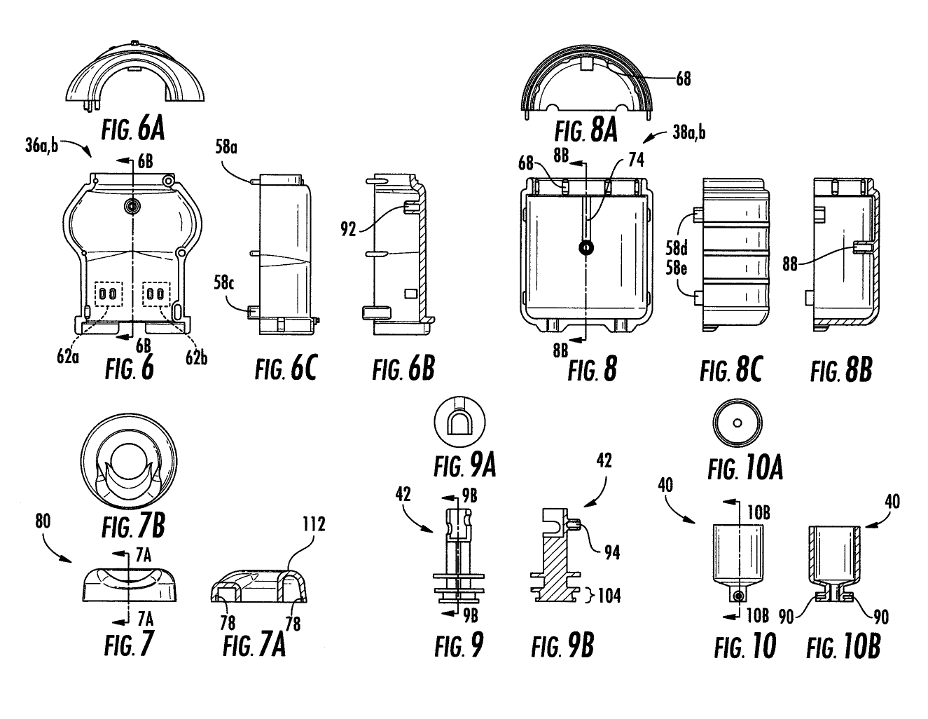

FIGS. 1, 2 and 3 illustrate the supplemental pain medication device 10 in its normal undepressed state. The amount of pain medication being supplied to the patient 16 is solely through the basal rate controller 14 when the supplemental pain medication device is on stand-by. In this state, a trigger 46 of the supplemental pain medication device 10 is in the normal undepressed state and the upper body 36 is stationary. In order to activate the supplemental pain medication device, the upper body 36 must be depressed into the lower body 38. To do so, the person (e.g., medical professional or patient 16) must first depress a trigger 46 or trumpet valve. Only after depressing the trigger 44 as shown in FIG. 4 is the upper body 36 allowed to be traversed or telescoped into the lower body 38 and the plunger 42 traversed further into the reservoir 40 to force more pain medication to the patient 16.

Referring now more particularly to FIGS. 3-5, the trigger 46 is securely attached to the plunger 42 by inserting the extensions 96a, b into the slots 98a, b of the plunger 42. The trigger 46 has an actuator 47 that slides within a body 49 of the trigger 46. The actuator 47 has a flow path 51 that is not aligned to the through holes formed in the extensions 96a, b when the actuator 47 is in the undepressed state (see FIG. 3). If the medical professional or patient 16 attempts to push the upper body 36 downward into the lower body 30a with the trigger 46 in the undepressed state, fluid in the reservoir 44 is not allowed to flow through the flow path 51 and out of the device 10. Thus, the upper body 36 is not allowed to be depressed into the lower body 38. However, when the actuator 47 is traversed to the depressed state (see FIG. 4), the flow path 51 is now aligned to the through holes formed in the extensions 96a, b to allow fluid within the reservoir 44 to flow out of the reservoir 44 through the tube 54, through the flow path 51 and out of tube 34. After the actuator 47 is traversed to the depressed state, fluid in the reservoir 44 is allowed to flow through the flow path 51 and out of the device 10. The plunger 42 can be depressed downward further into the reservoir 40.

When the plunger 42 is being traversed further into the reservoir 40, the fluid within the cavity 44 is pushed out of an opening 48 generally located at the bottom of the reservoir 40. This opening 48 is connected to bifurcation port 50 that is connected to tube 30 connected to the elastomeric pump 12 and also to tube 34 that is connected to the patient 16. The tube 30 upstream of the opening 48 and reservoir 44 may have a lumen defining an inner diameter 52. The tube(s) 34, 54 downstream of the opening 48 and reservoir 40 including but not limited to the tube 34 but also tube 54 may have a lumen defining an inner diameter 56. The inner diameter 52 of the tube 30 may be significantly smaller (e.g., twelve times smaller) than the inner diameter 56 of the tubes 54 and/or 34 so that fluid flowing through the bifurcation port 50 seeks the path of least resistance which would be through the tubes 54 and 34 instead of back into the tube 30. As such, pain medication contained within the inner cavity 44 is flowed out through the tube 54, into tube 34 and into the patient 16. Alternatively, instead of utilizing tubes having different inner diameters to provide a resistance differential, the tube 30 upstream of the reservoir 40 may have a flow restrictor that allows a significantly lower rate of fluid flow through the tube 30 compared to the rate of fluid flow through the tube 34 and/or 54 given the same amount of fluid pressure. In this regard when the plunger 42 is being traversed into the reservoir 40, the fluid within the inner cavity 44 of the reservoir 40 finds the path of least resistance which would be through the tubes 54 and 34 leading to the patient 16.

After depression of the plunger 42 into reservoir 40, the person releases the upper body 36 and the actuator 48. The actuator 47 traverses back to an undepressed state under force from a spring 53. The flow path 51 is misaligned to the throughholes of extension 96a, b so that fluid cannot flow through the flow path 51 and the device 10. At this time, the elastomeric pump 12 slowly forces fluid through the tube 30 and back into the inner cavity 44 of the reservoir. This also raises the plunger 42 back to the retracted position. Since the tube 30 or a flow restrictor incorporated into the tube 30 allows for a low flow rate through the tube 30, the inner cavity 44 is replenished with fluid or pain medication over a period of time (i.e., reset period). The tube 30 or flow restrictor may be sized so that the rest period is between one (1) minute and one (1) hour or a minute increment therebetween. As such, the patient 16 and/or medical professional cannot immediately depress the trigger 46 and upper body 36 in rapid succession in order to increase a sustained rate of pain medication to the patient 16 over a long period of time. The patient 16 and the medical professional must wait for the inner cavity 44 of the reservoir 40 to be filled up with pain medication. The pressure in the tube 30 pushes the plunger 42 back to the up position as shown in FIG. 3 and fills the inner cavity 44 of the reservoir 40 with pain medication fluid. The rest period may be changed by increasing or decreasing the size of the lumen of the tube upstream of the reservoir or by increasing or decreasing the restriction of the restrictor placed upstream of the reservoir to respectively increase or decrease the rest period.

Referring now to FIG. 5, an exploded perspective view of the device 10 is shown. The upper and lower bodies 36, 38 each comprise first and second halves 36a, b and 38a, b. The first and second halves 36a,b and 38a, b are identical to each other so that a single mold may be used to manufacture (e.g., injection molding) both the second and first halves 36a, b and 38a, b. More particularly, the first and second halves 36a, b may have protrusions 58a, b, c that are received into corresponding apertures 68a, b, c of the other half 36b, a for the purposes of holding the first and second halves 36a, b together. The interior side of the first and second halves 36a, b may have first and second clips 62a, b. These clips 62a, b hold the tubes 54, 34 in place as the upper body 36 is being telescoped into and out of the lower body 38. Only one of the set of clips 62a, b and one of the halves 36a, b is used to hold the tubes 54, 34. The other set of clips 62a, b in the other half of the upper body is unused.

The lower body 38 may have first and second halves 38a, b that are identical to each other. The first and second halves 38a, b may have protrusions 58d, e that are received into corresponding apertures 60d, e for the purposes of holding the first and second halves 38a, b together.

The upper body 36 may have a plurality of small rounded protrusions 64 that slidingly contact the inner surface 66 of the lower body 38. Similarly, the lower body 38 may have a plurality of small rounded protrusions 68 that slidingly contact the inner surface 70 of the upper body 36. The protrusions 64, 68 are staggered around collars of the upper and lower bodies 36, 38 to reduce the sliding friction between the upper and lower bodies 36, 38 as they telescoped within one another. Additionally, these protrusions 64, 68 align the central axis of the upper body 36 to the central axis of the lower body so that the linear traversal of the upper body 36 into and out of the lower body is along the central axes of the upper and lower bodies 36, 38.

Each of the halves 36a, b of the upper body 36 may have a tab 72. The tab 72 may ride within a groove 74 formed in the first and second halves 38a, b of the lower body 38. Each of the halves 36a, b of the upper body 36 may have a tab 76 that is received into an indented slot 78 of a cap 80 so that the cap 80 is aligned to the trigger 46 and the upper body 36.

The first and second halves 38a, b of the lower body 38 may have semicircular apertures 82 that collectively receive the tubes 30, 34. Preferably, the semicircular apertures 82a, b of the first and second halves 38a, b of the lower body 38 provide a friction fit with the tubes 30, 34 so that the tubes 30, 34 do not slide within the device 10 during actuation of the upper body 36 and the trigger 46.

The reservoir 40 remains fixed to the lower body 38, whereas the plunger 42 remains fixed to the upper body 36. As such, when the upper body 36 is telescoped into the lower body 38, the plunger 42 slides within the reservoir 40 to eject a supplemental amount of pain medication to the patient 16. The plunger 42 is only allowed to slide within the reservoir 40 to eject the supplemental amount of pain medication to the patient 16 when the actuator 47 of the trigger 46 is depressed to allow fluid to flow through the device 10. Otherwise, if the patient 16 or medical professional were to attempt to push the upper body 36 into the lower body 38 without depressing the trigger 46, the fluid within the reservoir 44 would not flow out of the device 10 and would not allow the upper body 36 to be telescoped into the lower body 30a or the plunger 42 to slide within the reservoir 40 to eject the supplemental amount of pain medication to the patient 16. The reservoir 40 and the plunger 42 are fixed to the lower and upper bodies 38, 36 by way of pins 84, 86. The first and second halves 38a, b of the lower body 38 may have apertures 88 that receive the pins 84. The pins 84 are also received into apertures 90 so that the reservoir 40 is locked in place. The reservoir 40 may rotate about the pins 84 in a limited fashion. The plunger 42 is fixed to the upper body 36 in a similar manner. More particularly, the pin 86 is received into aperture 92. However, instead of two pins 86, only one pin is received into one of the halves 36a, b of the upper body 36. The pin 86 is also received into an aperture 94 formed in the plunger 42. When the upper body 36 is traversed into the lower body 38, force is transferred through the pin 86 to the plunger 42 to also traverse the plunger 42 into the reservoir 40. Conversely, when fluid is being refilled into the inner cavity 44 of the reservoir 40, the pressure from the fluid transfers a force to the plunger 42 and back to the upper body 36 through the pin 86. The pin 86 allows for slight rotational movement of the plunger 42 so that any misalignment between the reservoir 40 and the plunger 42 may be resolved through the rotational movement of the plunger 42 and the reservoir 40.

The trigger 46 is mounted to the plunger 42 by placing extensions 96a, b into slots 98a, b. The trigger 46 is mounted so that an upper surface 110 of an actuator 47 of the trigger 46 is preferably slightly below an upper surface 112 of the cap 80, as shown in FIG. 3. If the upper body 36 is pushed against the wall or bed rail, the wall or bed rail would touch the upper surface 112 of the cap 80 instead of inadvertently depressing the actuator 47. Although the upper surface 110 of the actuator 47 of the trigger 46 is shown as being slightly below the upper surface 112 of the cap 80, it is also contemplated that the upper surface 110 of the actuator 47 of the trigger 46 may be at or even slightly above the upper surface 112 of the cap 80 yet still prevent inadvertent actuation of the upper body 36 into the lower body 38. If the upper surface 110 of the actuator 47 of the trigger is at the same level of the upper surface 112 of the cap 80, then the actuator 47 will not move if and when the upper body 36 is inadvertently pushed against a surface. If the upper surface 110 of the actuator 47 of the trigger is slightly above the upper surface 112 of the cap 80, then the actuator 47 may move slightly but not enough for the flow path 51 to align to the through holes in the extensions 96a, b.

The plunger 42 may have a gasket 100 that provides a circumferential seal with the inner surface of the reservoir 40. The gasket 100 may be fabricated from an isoprene material. Other materials are also contemplated such as rubber, polymeric, etc. The gasket 100 may have an inner cavity 102 that is sized and configured to receive and be snapped over a distal portion 104 of the plunger 42. The distal portion 104 of the plunger 42 fixes the orientation of the gasket 100 to the plunger 42. As the plunger 42 is moved up and down in the reservoir 40, the gasket increases and decreases the inner cavity 44 of the reservoir in order to eject pain medication fluid out of the device 10 and also to receive or replenish the pain medication fluid back into the inner cavity 44 of the reservoir 40. Axial alignment of the plunger 42 with the reservoir 40 may be accomplished with an upper disc of the plunger 42. The upper disc 106 does not form a liquid seal with the inner surface of the reservoir 40 but may slidingly contact the inner surface of the reservoir 40. The contact between the upper disc 106 and an inner surface as well as the gasket 100 and the inner surface of the reservoir 40 helps to maintain the axial alignment of the plunger 42 to the central axis of the reservoir 40.

Referring now to FIG. 2, in order to provide an additional amount of pain medication to the patient 16, the user may depress the trigger 46 which enables the fluidic pain medication in the reservoir 44 to flow out of the device 10 and allows the upper body 36 to slide into the lower body 38. When the upper body 36 is slid into the lower body 38, the plunger 42 slides within the reservoir 40 and ejects fluid or pain medication out of the reservoir 40. The fluid is ejected out of the reservoir 40 through bifurcation port 50. Since the tube 30 is significantly smaller than the tube 54 or the tube 30 is fitted with a restrictor so that the path of least resistance is through the tube 54 instead of the tube 30, the pain medication fluid flows out of the bifurcation port 50 into tube 54. Tube 54 is routed upward through the device 10 and into the extensions 96a, b and out through tube 34 to the patient 16.

The exterior surface of the upper body 36 may be provided with graduation marks 108. After depressing the trigger 46, the user then depresses the upper body 36. The user may regulate the amount of additional pain medication being supplied to the patient 16 by depressing the upper body 36 less than the full depression allowed and can determine the amount of additional pain medication by depressing the upper body 36 to a particular graduation mark 108.

The above description is given by way of example, and not limitation. Given the above disclosure, one skilled in the art could devise variations that are within the scope and spirit of the invention disclosed herein, including various ways of forming the upper and lower bodies 36, 38. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A device connectable to a pain medication delivery system for providing an additional amount of pain medication fluid to a patient when needed, the device comprising:
an inlet tube connectable to a pressurized supply line for receiving pain medication fluid into the device;
an outlet tube in fluid communication with the patient for providing pain medication fluid from the device to the patient;
a reservoir in fluid communication with the inlet tube and the outlet tube, the reservoir having;
a container for holding a volume of pain medication, the container in fluid communication with the inlet tube;
a plunger traversable between a retracted position and a depressed position and operative to flow pain medication fluid out of the outlet tube upon traversal of the plunger to the depressed position from the retracted position;
a trigger positioned between the reservoir and the outlet tube, the trigger having an actuator traversable between a depressed position and an undepressed position, the trigger having a flow path aligned to a fluid flow path of the outlet tube to allow the pain medication in the container to flow through the outlet tube when the actuator is in the depressed position, the flow path being misaligned to the flow path of the outlet tube to stop the pain medication in the container from flowing through the outlet tube when the actuator is in the undepressed position.

2. The device of claim 1 wherein:
the trigger further comprises a body having first and second apertures;
the device further comprises a connecting tube in fluid communication with the container and the first aperture of the body of the trigger;
the outlet tube being in fluid communication with the second aperture of the body of the trigger;
wherein the flow path of the trigger is formed in the actuator, the flow path of the actuator is aligned to the first and second apertures of the body of the trigger when the actuator is in the undepressed position, the flow path of the actuator being misaligned to the first and second apertures of the body of the trigger when the actuator is in the depressed position.

3. The device of claim 1 wherein the container is attached to a first body and the plunger is attached to a second body, the first and second bodies capable of being telescoped into each other for sliding the plunger in the container.

4. The device of claim 3 further comprising a cap attached to the second body, an exposed surface of the actuator of the trigger being positioned below an exposed surface of the cap to prevent inadvertent depression of the actuator.

5. The device of claim 1 wherein flow upstream of the reservoir is more restricted than flow downstream of the reservoir so that upon depression of the plunger into the container, the pain medication fluid in the container flows downstream instead of upstream.

6. The device of claim 1 wherein a lumen of the inlet tube is smaller than a lumen of the connecting tube and the outlet tube for restricting flow upstream of the reservoir more than flow downstream of the reservoir.

7. The device of claim 1 wherein a flow restrictor is mounted upstream of the reservoir so that upon depression of the plunger into the container, fluid flows upstream.

8. A method of operating a secondary device for providing an additional amount of pain medication fluid to a patient when needed, the method comprising the steps of:
connecting the secondary device parallel to a primary pain medication device, the secondary device having zero flow of pain medication fluid until actuated;
depressing an actuator of a trigger to establish fluid communication between inlet and outlet tubes to enable flow of additional paid medication fluid out of the secondary device; and
depressing first and second bodies with respect to each other to flow pain medication fluid out of the secondary device.

9. The method of claim 8 wherein the depressing step aligns a flow path of the actuator to a flow of the outlet tube.

10. The method of claim 8 wherein the depressing step establishes fluid communication between the inlet and outlet tubes.
